SEQUENCE LISTING (16 sequences)

SEQ ID NO: 1 - protein, 692 aa, Homo sapiens
MGTVSSRRSWWPLPLLLLLLLLLGPAGARAQEDEDGDYEELVLALRSEEDGLAEAPEHGTTATFHRCAKDPWRLPGTYVVVLKEETHLSQSERTARRLQAQAARRGYLTKILHVFHGLLPGFLVKMSGDLLELALKLPHVDYIEEDSSVFAQSIPWNLERITPPRYRADEYQPPDGGSLVEVYLLDTSIQSDHREIEGRVMVTDFENVPEEDGTRFHRQASKCDSHGTHLAGVVSGRDAGVAKGASMRSLRVLNCQGKGTVSGTLIGLEFIRKSQLVQPVGPLVVLLPLAGGYSRVLNAACQRLARAGVVLVTAAGNFRDDACLYSPASAPEVITVGATNAQDQPVTLGTLGTNFGRCVDLFAPGEDIIGASSDCSTCFVSQSGTSQAAAHVAGIAAMMLSAEPELTLAELRQRLIHFSAKDVINEAWFPEDQRVLTPNLVAALPPSTHGAGWQLFCRTVWSAHSGPTRMATAVARCAPDEELLSCSSFSRSGKRRGERMEAQGGKLVCRAHNAFGGEGVYAIARCCLLPQANCSVHTAPPAEASMGTRVHCHQQGHVLTGCSSHWEVEDLGTHKPPVLRPRGQPNQCVGHREASIHASCCHAPGLECKVKEHGIPAPQEQVTVACEEGWTLTGCSALPGTSHVLGAYAVDNTCVVRSRDVSTTGSTSEGAVTAVAICCRSRHLAQASQELQ

SEQ ID NO: 2 - protein, 118 aa, Artificial
  Synthetic Construct
QVQLVQSGAEVKKPGASVKVSCKASGYTFTSYYMHWVRQAPGQGLEWMGEISPFGGRTNYNEKFKSRVTMTRDTSTSTVYMELSSLRSEDTAVYYCARERPLYASDLWGQGTTVTVSS

SEQ ID NO: 3 - protein, 107 aa, Artificial
  Synthetic Construct
DIQMTQSPSSLSASVGDRVTITCRASQGISSALAWYQQKPGKAPKLLIYSASYRYTGVPSRFSGSGSGTDFTFTISSLQPEDIATYYCQQRYSLWRTFGQGTKLEIK

SEQ ID NO: 4 - protein, 5 aa, Artificial
  Synthetic Construct
SYYMH

SEQ ID NO: 5 - protein, 7 aa, Artificial
  Synthetic Construct
GYTFTSY

SEQ ID NO: 6 - protein, 10 aa, Artificial
  Synthetic Construct
GYTFTSYYMH

SEQ ID NO: 7 - protein, 17 aa, Artificial
  Synthetic Construct
EISPFGGRTNYNEKFKS

SEQ ID NO: 8 - protein, 6 aa, Artificial
  Synthetic Construct
SPFGGR

SEQ ID NO: 9 - protein, 9 aa, Artificial
  Synthetic Construct
ERPLYASDL

SEQ ID NO: 10 - protein, 11 aa, Artificial
  Synthetic Construct
RASQGISSALA

SEQ ID NO: 11 - protein, 7 aa, Artificial
  Synthetic Construct
SASYRYT

SEQ ID NO: 12 - protein, 9 aa, Artificial
  Synthetic Construct
QQRYSLWRT

SEQ ID NO: 13 - protein, 214 aa, Artificial
  Synthetic Construct
DIQMTQSPSSLSASVGDRVTITCRASQGISSALAWYQQKPGKAPKLLIYSASYRYTGVPSRFSGSGSGTDFTFTISSLQPEDIATYYCQQRYSLWRTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 14 - protein, 444 aa, Artificial
  Synthetic Construct
QVQLVQSGAEVKKPGASVKVSCKASGYTFTSYYMHWVRQAPGQGLEWMGEISPFGGRTNYNEKFKSRVTMTRDTSTSTVYMELSSLRSEDTAVYYCARERPLYASDLWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPSSIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 15 - protein, 40 aa, Homo sapiens
GTNECLDNNGGCSHVCNDLKIGYECLCPDGFQLVAQRRCE

SEQ ID NO: 16 - protein, 860 aa, Homo sapiens [sequence omitted]

(19) United States
(12) Patent Application Publication
(10) Pub. No.: US 2017/0224816 A1
(43) Pub. Date: Aug. 10, 2017

(54) METHODS FOR REDUCING LDL-CHOLESTEROL

(71) Applicant: RINAT NEUROSCIENCE CORP., SOUTH SAN FRANCISCO, CA (US)

(72) Inventors: Rachel Marie DeVay JACOBSON, Belmont, CA (US); Hong LIANG, Hillsborough, CA (US); David Shelton, Oakland, CA (US)

(73) Assignee: RINAT NEUROSCIENCE CORP., SOUTH SAN FRANCISCO, CA (US)

(21) Appl. No.: 15/501,752

(22) PCT Filed: Jul. 29, 2015
(86) PCT No.: PCT/IB2015/055732

§ 371 Date: Feb. 3, 2017

Related U.S. Application Data
(60) Provisional application No. 62/034,021, filed on Aug. 6, 2014.

(51) Int. Cl. A61K 39/395; A61K 31/22; A61K 31/405; A61K 39/29; A61K 31/365

(52) U.S. Cl. CPC A61K 39/3955; A61K 39/292; A61K 31/365; A61K 31/405; A61K 31/22; C07K 2317/76; A61K 2300/00

(57) ABSTRACT

The present invention relates to methods for the treatment of reducing LDL-cholesterol levels in a subject infected with hepatitis C virus (HCV) or at high risk of contracting HCV comprising administration to the subject in need thereof a therapeutically effective amount of an antagonist antibody which specifically binds to a human PCSK9 protein. The subject treatment can be used in the prevention and/or treatment of cholesterol and lipoprotein metabolism disorders, including hypercholesterolemia, dyslipidemia, hyperlipidemia, atherosclerosis, acute coronary syndrome and, more generally, cardiovascular disease (CVD).

### CROSS-REFERENCE TO RELATED APPLICATION

This application claims the benefits of U.S. provisional application No. 62/034,021 filed Aug. 6, 2014, which is hereby incorporated by reference in its entirety.

### FIELD

The present invention relates to methods or uses for reducing LDL-cholesterol levels in a subject infected with hepatitis C virus (HCV) or at high risk of contracting HCV comprising administration to the subject in need thereof a therapeutically effective amount of an antagonist antibody which specifically binds to a human PCSK9 protein. The subject treatment can be used in the prevention and/or treatment of cholesterol and lipoprotein metabolism disorders in a subpopulation of patients infected with HCV, including hypercholesterolemia, dyslipidemia, hyperlipidemia, atherosclerosis, acute coronary syndrome and, more generally, cardiovascular disease (CVD).

### BACKGROUND

Proprotein convertase subtilisin/kexin type 9 (PCSK9) has recently become recognized as a key player in regulating cholesterol metabolism and has emerged as a promising target for prevention and treatment of coronary heart disease (CHD) (see, e.g., Seidah et al., Proc. Natl. Acad. Sci. U.S.A 100:928-33, 2003). Gain-of-function (GOF) mutations in PCSK9 have been found to be associated with autosomal dominant hypercholesterolaemia (ADH) (see, e.g., Abifadel et al., Nat. Genet. 34:154-6, 2003), mild to severe hypercholesterolaemia, and an increased risk of CHD (see, e.g., Davignon et al., Curr. Atheroscler. Rep. 12:308-15, 2010). Conversely, the loss-of-function (LOF) mutations in PCSK9 are associated with lifelong reductions in low-density lipoprotein cholesterol (LDL-C) (see, e.g., Cohen et al., Nat. Genet. 37:161-5, 2005; and Tibolla et al., Nutr. Metab. Cardiovasc. Dis. 21:835-43, 2011). Further, the LOF mutations in PCSK9 have been found to reduce the atherosclerosis and CHD risk (see, e.g., Cohen et al., N Eng J Med 354: 1264-72, 2010; Benn et al., J Am Coll Cardiol 55:2833-42, 2010); whereas the complete loss of PCSK9 results in low serum LDL-C of <20 mg/dl in human health subjects (Hooper et al., Atheroscler. 193:445-8, 2007; and Zhao et al., Am. J. Hum. Genet. 79: 514-23, 2006).

The main way by which PCSK9 regulates LDL-C levels is modulating the degradation of the LDL receptor (LDLR) by direct interaction with the LDLR both within the cell and at the surface of the plasma membrane (see, e.g., Seidah et al., Nat. Rev. Drug. Discov. 11:367-83, 2012; and Lambert et al., J. Lipid. Res. 53:2515-24, 2012). Highly expressed in the liver and intestine, PCSK9 is secreted after the autocatalytic cleavage of the prodomain and can bind to the LDLR in a complex, which triggers modification of LDLR conformation, avoiding the normal recycling of LDLR to the plasma membrane, and increasing LDLR lysosomal degradation (see e.g., Horton et al, J. Lipid. Res. 50:S172-S177, 2009; Piper et al., Structure 15:545-52, 2007; and Lo Surdo et al., EMBO Rep. 12:1300-5, 2011).

Recent studies have shown that, in addition to LDLR, PCSK9 can also down-regulate the cell surface expression of CD81, which is a major hepatitis C virus (HCV) receptor and that circulating liver PCSK9 has an antiviral effect on HCV in cells (see, e.g., Labonté et al., Heptology 50:17-24, 2009). U.S. Pat. No. 8,088,571 further discloses a method of treating and/inhibiting PCSK9-susceptible viral infection (e.g., HCV) comprising decreasing the expression of CD81 at the surface of cells by increasing PCSK9 activity and/or expression.

All publications, patents, and patent applications cited herein are hereby incorporated by reference herein in their entirety for all purposes to the same extent as if each individual publication, patent, and patent application were specifically and individually indicated to be so incorporated by reference. In the event that one or more of the incorporated literature and similar materials differs from or contradicts this application, including but not limited to defined terms, term usage, described techniques, or the like, this application controls

### SUMMARY

The present invention relates to methods for reducing LDL-cholesterol levels in blood of a subject infected with hepatitis C virus (HCV) or at high risk of contracting HCV, comprising administering to the subject in need thereof a therapeutically effective amount of an antagonist antibody which specifically binds to the human PCSK9 protein.

The inventors have discovered that, while there is a dose dependent loss of CD81 and LDLR in the presence of PCSK9, administration of a PCSK9 antibody that antagonizes the PCSK9 interaction with the LDLR can restore LDLR levels and surprisingly has no effect on PCSK9 mediated CD81 degradation, indicating that PCSK9 degrades CD81 via a unique epitope that is distinct from the epitope involved in LDLR binding and degradation. Thus, administration of an antagonist antibody that inhibits the PCSK9 effect on LDLR and cholesterol levels does not necessarily lead to increased CD81 levels and increased HCV viral infection.

Accordingly, in one aspect, this invention provides a method of reducing a level of LDL-cholesterol in blood of a subject infected with HCV or at high risk of contracting HCV, comprising administering to the subject in need thereof a therapeutically effective amount of an antagonist antibody which specifically binds to a human proprotein convertase subtilisin kexin type 9 (PCSK9) of SEQ ID NO:1.

In some embodiments, provided is an antagonist antibody which specifically binds to a human PCSK9 of SEQ ID NO:1 for use in reducing a level of LDL-cholesterol in blood of a subject infected with HCV or at high risk of contracting HCV.

In some embodiments, provided is a use of an antagonist antibody which specifically binds to a human PCSK9 of SEQ ID NO:1 in the manufacture of a medicament for reducing a level of LDL-cholesterol in blood of a subject infected with HCV or at high risk of contracting HCV.

In some embodiments, the anti-PCSK9 antibody blocks LDLR binding to the PCSK9 antibody of SEQ ID NO: 1. In some embodiments, the anti-PCSK9 antibody is alirocumab (PRALUENT™), evolocumab (REPATHA™), REGN728, LGT209, RG7652, LY3015014, L1L3, 31H4, 11F1, 12H11, 8A1, 8A3, 3C4, or 1D05. In some embodiments, the anti-PCSK9 antibody is a full antagonist of the PCSK9-mediated effect LDL receptor (LDLR) levels as measured in vitro using an LDLR down-regulation assay in Huh7 cells. In some embodiments, the anti-PCSK9 antibody comprises a heavy chain variable region (VH) comprising complementarity determining region one (CDR1), CDR2, and CDR3 of the amino acid sequence shown in SEQ ID NO: 2; and a light chain variable region (VL) comprising CDR1, CDR2, and CDR3 of the amino acid sequence shown in SEQ ID NO: 3. In some embodiments, the anti-PCSK9 antibody comprises a VH CDR1 having the amino acid sequence shown in SEQ ID NO: 4, 5, or 6, a VH CDR2 having the amino acid sequence shown in SEQ ID NO:7 or 8, a VH CDR3 having the amino acid sequence shown in SEQ ID NO: 9, a VL CDR1 having the amino acid sequence shown in SEQ ID NO:10, a VL CDR2 having the amino acid sequence shown in SEQ ID NO:11, and a VL CDR3 having the amino acid sequence shown in SEQ ID NO: 12. In some embodiments, the anti-PCSK9 antibody comprises a light chain having SEQ ID NO: 13 and a heavy chain having SEQ ID NO: 14, with or without the C-terminal lysine of SEQ ID NO: 14.

In some embodiments, the method described herein comprises administering about 10 mg to about 2000 mg of the anti-PCSK9 antibody to the subject, for example, intravenously or subcutaneously. In some embodiments, the anti-PCSK9 antibody is administered at least every four weeks or every 2 weeks to the subject.

In some embodiments, an antiviral therapy has been administered prior to the initial dose of the antibody.

In some embodiments, a statin can be administered prior to the initial dose of the anti-PCSK antibody. In some embodiments, a daily dose of a statin is administered. In other embodiments, stable doses of the statin have been administered for at least about two, three, four, five, or six weeks prior to the initial dose of the anti-PCSK9 antibody. Examples of a statin include atorvastatin, cerivastatin, fluvastatin, lovastatin, mevastatin, pitavastatin, pravastatin, rosuvastatin, simvastatin, or any pharmaceutically acceptable salts, or stereoisomers thereof.

The method described herein can be used for treating or prophylactically treating a subject infected with HCV or at high risk of contracting HCV and suffering from dyslipidemia, hyperlipidemia, hypercholesterolemia, atherosclerosis, cardiovascular disease, and/or coronary heart disease.

In some embodiments, provided is a use of a PCSK9 antagonist antibody in a method of the invention, as set forth in any one of the preceding embodiments.

In some embodiments, provided is a use of a PCSK9 antagonist antibody in a method of manufacture of a medicament for use in a method as set forth in any one of the preceding embodiments.

In some embodiments, provided is a PCSK9 antagonist antibody for use as set forth in any one of the preceding embodiments.

In another aspect, this invention provides a kit or an article of manufacture, comprising a container, a composition within the container comprising a PCSK9 antagonist antibody, and a package insert containing instructions to administer a therapeutically effective amount of the PCSK9 antagonist antibody for reducing a level of LDL-cholesterol in blood of a subject infected with HCV or at high risk of contracting HCV.

### BRIEF DESCRIPTION OF THE FIGURES/DRAWINGS

FIG. 1 depicts the protein expression (by Western blot) of CD81, LDLR, and beta-actin in liver lysate from mice treated with PCSK9 antagonist antibody 5A10 and antibody isotype control.

FIG. 2 illustrates the quantification of signal intensities of CD81 and LDLR from Western blot analysis in liver lysate from mice treated with PCSK9 antagonist antibody 5A10 and antibody isotype control (n=3/group).

FIG. 3 illustrates percent average relative fluorescence of CD81 levels from at least 50 cells from each treatment (3A) and depicts surface staining of LDLR and CD81 on DAPI stained HepG2 cells following treatment with 300 nM PCSK9 together with 300 nM of isotype control antibody (IC) or 300 nM of PCSK9 together with 300 nM PCSK9 antagonist antibody L1L3 (3B).

FIG. 4 depicts the protein expression of LDLR, CD81, and TFNR in HepG2 cells treated with increasing concentrations of PCSK9 in the presence of isotype control antibody (IC) or PCSK9 antagonist antibody L1L3 (4A) and illustrates the quantification of normalized signal intensities of LDLR and CD81 (4B). Integrated intensity of CD81 or LDLR from each lane was normalized to TFNR and taken as a percentage of untreated cells.

### DETAILED DESCRIPTION

The present invention relates to methods of reducing LDL-cholesterol levels in blood of a subject infected with hepatitis C virus (HCV) or at high risk of contracting HCV comprising administering to the subject a therapeutically effective amount of an antagonist antibody which specifically binds to the human PCSK9 protein. The inventors have discovered that, while there is a dose dependent loss of CD81 and LDLR in the presence of PCSK9, administration of a PCSK9 antibody that antagonizes the PCSK9 interaction with the LDLR can restore LDLR levels and surprisingly has no effect on PCSK9 mediated CD81 degradation, indicating that PCSK9 degrades CD81 via a unique epitope that is distinct from the epitope involved in LDLR binding and degradation. Thus, administration of an antagonist antibody that inhibits the PCSK9 effect on LDLR and cholesterol levels does not necessarily lead to increased CD81 levels and increased HCV viral infection. The methods described herein can be used in the prevention and/or treatment of cholesterol and lipoprotein metabolism disorders in a subpopulation of patients infected with HCV or at high risk of contracting HCV, such as hypercholesterolemia (e.g., heterozygous familial hypercholesterolemia (HetFH) or homozygous familial hypercholesterolemia (HoFH)), dyslipidemia (e.g., mixed dyslipidemia), hyperlipidemia (e.g., heterozygous or homozygous familial and non-familial), atherosclerosis, acute coronary syndrome and, more generally, and cardiovascular disease (CVD).

### General Techniques

The practice of the present invention will employ, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, second edition (Sambrook et al., 1989) Cold Spring Harbor Press; Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-1998) J. Wiley and Sons; Methods in Enzymology (Academic Press, Inc.); Handbook of Experimental Immunology (D. M. Weir and C. C. Blackwell, eds.); Gene Transfer Vectors for Mammalian Cells (J. M. Miller and M. P. Calos, eds., 1987); Current Protocols in Molecular Biology (F. M. Ausubel et al., eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practical approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds., Harwood Academic Publishers, 1995).

### Definition

An “antibody” is an immunoglobulin molecule capable of specific binding to a target, such as a carbohydrate, polynucleotide, lipid, polypeptide, etc., through at least one antigen recognition site, located in the variable region of the immunoglobulin molecule. As used herein, the term “antibody” encompasses not only intact polyclonal or monoclonal antibodies, but also any antigen binding fragment (i.e., “antigen-binding portion”) or single chain thereof, fusion proteins comprising an antibody, and any other modified configuration of the immunoglobulin molecule that comprises an antigen recognition site including, for example without limitation, scFv, single domain antibodies (e.g., shark and camelid antibodies), maxibodies, minibodies, intrabodies, diabodies, triabodies, tetrabodies, v-NAR and bis-scFv (see, e.g., Hollinger and Hudson, 2005, Nature Biotechnology 23(9): 1126-1136). An antibody includes an antibody of any class, such as IgG, IgA, or IgM (or sub-class thereof), and the antibody need not be of any particular class. Depending on the antibody amino acid sequence of the constant region of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The heavy-chain constant regions that correspond to the different classes of immunoglobulins are called alpha, delta, epsilon, gamma, and mu, respectively. The subunit structures and three-dimensional configurations of different classes of immunoglobulins are well known.

The term “antigen binding portion” or “antigen binding fragment” of an antibody, as used herein, refers to one or more fragments of an intact antibody that retain the ability to specifically bind to a given antigen (e.g., PCSK9). Antigen binding functions of an antibody can be performed by fragments of an intact antibody. Examples of binding fragments encompassed within the term “antigen binding portion” of an antibody include Fab; Fab′; F(ab′)₂; an Fd fragment consisting of the VH and CH1 domains; an Fv fragment consisting of the VL and VH domains of a single arm of an antibody; a single domain antibody (dAb) fragment (Ward et al., 1989, Nature 341:544-546), and an isolated complementarity determining region (CDR).

The term “monoclonal antibody” (Mab) refers to an antibody that is derived from a single copy or clone, including e.g., any eukaryotic, prokaryotic, or phage clone, and not the method by which it is produced. Preferably, a monoclonal antibody of the invention exists in a homogeneous or substantially homogeneous population.

“Humanized” antibody refers to forms of non-human (e.g. murine) antibodies that are chimeric immunoglobulins, immunoglobulin chains, or fragments thereof (such as Fv, Fab, Fab′, F(ab′)₂ or other antigen-binding subsequences of antibodies) that contain minimal sequence derived from non-human immunoglobulin. Preferably, humanized antibodies are human immunoglobulins (recipient antibody) in which residues from a complementary determining region (CDR) of the recipient are replaced by residues from a CDR of a non-human species (donor antibody) such as mouse, rat, or rabbit having the desired specificity, affinity, and capacity.

As used herein, “human antibody” means an antibody having an amino acid sequence corresponding to that of an antibody that can be produced by a human and/or which has been made using any of the techniques for making human antibodies known to those skilled in the art or disclosed herein. This definition of a human antibody includes antibodies comprising at least one human heavy chain polypeptide or at least one human light chain polypeptide. One such example is an antibody comprising murine light chain and human heavy chain polypeptides. Human antibodies can be produced using various techniques known in the art. In one embodiment, the human antibody is selected from a phage library, where that phage library expresses human antibodies (Vaughan et al., 1996, Nature Biotechnology, 14:309-314; Sheets et al., 1998, Proc. Natl. Acad. Sci. (USA) 95:6157-6162; Hoogenboom and Winter, 1991, J. Mol. Biol., 227:381; Marks et al., 1991, J. Mol. Biol., 222:581). Human antibodies can also be made by immunization of animals into which human immunoglobulin loci have been transgenically introduced in place of the endogenous loci, e.g., mice in which the endogenous immunoglobulin genes have been partially or completely inactivated. This approach is described in U.S. Pat. Nos. 5,545,807; 5,545,806; 5,569,825; 5,625,126; 5,633,425; and 5,661,016. Alternatively, the human antibody may be prepared by immortalizing human B lymphocytes that produce an antibody directed against a target antigen (such B lymphocytes may be recovered from an individual or may have been immunized in vitro). See, e.g., Cole et al. Monoclonal Antibodies and Cancer Therapy, Alan R. Liss, p. 77, 1985; Boerner et al., 1991, J. Immunol., 147 (1):86-95; and U.S. Pat. No. 5,750,373.

A “bispecific,” “dual-specific” or “bifunctional” antibody is a hybrid antibody having two different antigen binding sites. The two antigen binding sites of a bispecific antibody bind to two different epitopes, which may reside on the same or different protein targets (e.g., PCSK9 protein).

A “variable region” of an antibody refers to the variable region of the antibody light chain or the variable region of the antibody heavy chain, either alone or in combination. As known in the art, the variable regions of the heavy and light chain each consist of four framework regions (FRs) connected by three complementarity determining regions (CDRs) also known as hypervariable regions, contribute to the formation of the antigen binding site of antibodies. If variants of a subject variable region are desired, particularly with substitution in amino acid residues outside of a CDR region (i.e., in the framework region), appropriate amino acid substitution, preferably, conservative amino acid substitution, can be identified by comparing the subject variable region to the variable regions of other antibodies which contain CDR1 and CDR2 sequences in the same canonical class as the subject variable region (Chothia and Lesk, J. Mol. Biol. 196(4): 901-917, 1987). When choosing FR to flank subject CDRs, e.g., when humanizing or optimizing an antibody, FRs from antibodies which contain CDR1 and CDR2 sequences in the same canonical class are preferred.

A “CDR” of a variable domain are amino acid residues within the variable region that are identified in accordance with the definitions of the Kabat, Chothia, the accumulation of both Kabat and Chothia, AbM, contact, and/or conformational definitions or any method of CDR determination well known in the art. Antibody CDRs may be identified as the hypervariable regions originally defined by Kabat et al. See, e.g., Kabat et al., 1992, Sequences of Proteins of Immunological Interest, 5th ed., Public Health Service, NIH, Washington D.C. The positions of the CDRs may also be identified as the structural loop structures originally described by Chothia and others. See, e.g., Chothia et al., 1989, Nature 342:877-883. Other approaches to CDR identification include the “AbM definition,” which is a compromise between Kabat and Chothia and is derived using Oxford Molecular's AbM antibody modeling software (now) ACCELRYS®), or the “contact definition” of CDRs based on observed antigen contacts, set forth in MacCallum et al., 1996, J. Mol. Biol., 262:732-745. In another approach, referred to herein as the “conformational definition” of CDRs, the positions of the CDRs may be identified as the residues that make enthalpic contributions to antigen binding. See, e.g., Makabe et al., 2008, Journal of Biological Chemistry, 283:1156-1166. Still other CDR boundary definitions may not strictly follow one of the above approaches, but will nonetheless overlap with at least a portion of the Kabat CDRs, although they may be shortened or lengthened in light of prediction or experimental findings that particular residues or groups of residues or even entire CDRs do not significantly impact antigen binding. As used herein, a CDR may refer to CDRs defined by any approach known in the art, including combinations of approaches. The methods used herein may utilize CDRs defined according to any of these approaches. For any given embodiment containing more than one CDR, the CDRs may be defined in accordance with any of Kabat, Chothia, extended, AbM, contact, and/or conformational definitions.

As known in the art a “constant region” of an antibody refers to the constant region of the antibody light chain or the constant region of the antibody heavy chain, either alone or in combination.

As used herein, the term “PCSK9” refers to any form of PCSK9 and variants thereof that retain at least part of the activity of PCSK9. Unless indicated differently, such as by specific reference to human PCSK9, PCSK9 includes all mammalian species of native sequence PCSK9, e.g., human, canine, feline, equine, and bovine. One exemplary human PCSK9 is found as Uniprot Accession Number Q8NBP7 (SEQ ID NO: 1).

As used herein, an “anti-PCSK9 antagonist antibody” or “PCSK9 antagonist antibody” refers to an anti-PCSK9 antibody that is able to inhibit PCSK9 biological activity and/or downstream pathway(s) mediated by PCSK9 signaling, including PCSK9-mediated down-regulation of the LDLR, and PCSK9-mediated decrease in LDL blood clearance. A PCSK9 antagonist antibody encompasses antibodies that block, antagonize, suppress or reduce (to any degree including significantly) PCSK9 biological activity, including downstream pathways mediated by PCSK9 signaling, such as LDLR interaction, or elicitation of a cellular response to PCSK9. For purpose of the present invention, it will be explicitly understood that the term “PCSK9 antagonist antibody” encompasses all the previously identified terms, titles, and functional states and characteristics whereby the PCSK9 itself, a PCSK9 biological activity (including but not limited to its ability to mediate any aspect of interaction with the LDLR, down regulation of LDLR, and decreased blood LDL clearance), or the consequences of the biological activity, are substantially nullified, decreased, or neutralized in any meaningful degree. In some embodiments, a PCSK9 antagonist antibody binds PCSK9 and prevents interaction with the LDLR. Examples of PCSK9 antagonist antibodies are provided in, e.g., U.S. Patent Application Publication No. 20100068199 and Devay et al., J. Biol. Chem. 288: 10805-10818 (2013), which are herein incorporated by reference in its entirety.

As used herein, a “full antagonist” is an antagonist which, at an effective concentration, essentially completely blocks a measurable effect of PCSK9. By a partial antagonist is meant an antagonist that is capable of partially blocking a measurable effect, but that, even at a highest concentration is not a full antagonist. By essentially completely is meant at least about 80%, preferably, at least about 90%, more preferably, at least about 95%, and most preferably, at least about 98% or 99% of the measurable effect is blocked. The relevant “measurable effects” are described herein and include down regulation of LDLR by a PCSK9 antagonist as assayed in Huh7 cells in vitro, in vivo decrease in blood (or plasma) levels of total cholesterol, and in vivo decrease in LDL levels in blood (or plasma).

The term “epitope” refers to that portion of a molecule capable of being recognized by and bound by an antibody at one or more of the antibody's antigen-binding regions. Epitopes often consist of a surface grouping of molecules such as amino acids or sugar side chains and have specific three-dimensional structural characteristics as well as specific charge characteristics. In some embodiments, the epitope can be a protein epitope. Protein epitopes can be linear or conformational. In a linear epitope, all of the points of interaction between the protein and the interacting molecule (such as an antibody) occur linearly along the primary amino acid sequence of the protein. A “nonlinear epitope” or “conformational epitope” comprises noncontiguous polypeptides (or amino acids) within the antigenic protein to which an antibody specific to the epitope binds. The term “antigenic epitope” as used herein, is defined as a portion of an antigen to which an antibody can specifically bind as determined by any method well known in the art, for example, by conventional immunoassays. Once a desired epitope on an antigen is determined, it is possible to generate antibodies to that epitope, e.g., using the techniques described in the present specification. Alternatively, during the discovery process, the generation and characterization of antibodies may elucidate information about desirable epitopes. From this information, it is then possible to competitively screen antibodies for binding to the same epitope. An approach to achieve this is to conduct competition and cross-competition studies to find antibodies that compete or cross-compete with one another for binding to PCSK9, e.g., the antibodies compete for binding to the antigen.

As used herein, the term “clinically meaningful” means at least a 15% reduction in blood LDL-cholesterol levels in humans or at least a 15% reduction in total blood cholesterol in mice. It is clear that measurements in plasma or serum can serve as surrogates for measurement of levels in blood.

The terms “polypeptide”, “oligopeptide”, “peptide” and “protein” are used interchangeably herein to refer to chains of amino acids of any length, preferably, relatively short (e.g., 10-100 amino acids). The chain may be linear or branched, it may comprise modified amino acids, and/or may be interrupted by non-amino acids. The terms also encompass an amino acid chain that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. It is understood that the polypeptides can occur as single chains or associated chains.

As known in the art, “polynucleotide,” or “nucleic acid,” as used interchangeably herein, refer to chains of nucleotides of any length, and include DNA and RNA. The nucleotides can be deoxyribonucleotides, ribonucleotides, modified nucleotides or bases, and/or their analogs, or any substrate that can be incorporated into a chain by DNA or RNA polymerase. A polynucleotide may comprise modified nucleotides, such as methylated nucleotides and their analogs. If present, modification to the nucleotide structure may be imparted before or after assembly of the chain. The sequence of nucleotides may be interrupted by non-nucleotide components. A polynucleotide may be further modified after polymerization, such as by conjugation with a labeling component. Other types of modifications include, for example, “caps”, substitution of one or more of the naturally occurring nucleotides with an analog, internucleotide modifications such as, for example, those with uncharged linkages (e.g., methyl phosphonates, phosphotriesters, phosphoamidates, carbamates, etc.) and with charged linkages (e.g., phosphorothioates, phosphorodithioates, etc.), those containing pendant moieties, such as, for example, proteins (e.g., nucleases, toxins, antibodies, signal peptides, poly-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-, 2′-O-allyl, 2′-fluoro- or 2′-azido-ribose, carbocyclic sugar analogs, alpha- or beta-anomeric sugars, epimeric sugars such as arabinose, xyloses or lyxoses, pyranose sugars, furanose sugars, sedoheptuloses, acyclic analogs and abasic nucleoside analogs such as methyl riboside. One or more phosphodiester linkages may be replaced by alternative linking groups. These alternative linking groups include, but are not limited to, embodiments wherein phosphate is replaced by P(O)S(“thioate”), P(S)S (“dithioate”), (O)NR₂ (“amidate”), P(O)R, P(O)OR′, CO or CH₂ (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. The preceding description applies to all polynucleotides referred to herein, including RNA and DNA.

An antibody that “preferentially binds” or “specifically binds” (used interchangeably herein) to an epitope is a term well understood in the art, and methods to determine such specific or preferential binding are also well known in the art. A molecule is said to exhibit “specific binding” or “preferential binding” if it reacts or associates more frequently, more rapidly, with greater duration and/or with greater affinity with a particular cell or substance than it does with alternative cells or substances. An antibody “specifically binds” or “preferentially binds” to a target if it binds with greater affinity, avidity, more readily, and/or with greater duration than it binds to other substances. For example, an antibody that specifically or preferentially binds to a PCSK9 epitope is an antibody that binds this epitope with greater affinity, avidity, more readily, and/or with greater duration than it binds to other PCSK9 epitopes or non-PCSK9 epitopes. It is also understood by reading this definition that, for example, an antibody (or moiety or epitope) that specifically or preferentially binds to a first target may or may not specifically or preferentially bind to a second target. As such, “specific binding” or “preferential binding” does not necessarily require (although it can include) exclusive binding. Generally, but not necessarily, reference to binding means preferential binding.

As used herein, “substantially pure” refers to material which is at least 50% pure (i.e., free from contaminants), more preferably, at least 90% pure, more preferably, at least 95% pure, yet more preferably, at least 98% pure, and most preferably, at least 99% pure.

A “host cell” includes an individual cell or cell culture that can be or has been a recipient for vector(s) for incorporation of polynucleotide inserts. Host cells include progeny of a single host cell, and the progeny may not necessarily be completely identical (in morphology or in genomic DNA complement) to the original parent cell due to natural, accidental, or deliberate mutation. A host cell includes cells transfected in vivo with a polynucleotide(s) of this invention.

As known in the art, the term “Fc region” is used to define a C-terminal region of an immunoglobulin heavy chain. The “Fc region” may be a native sequence Fc region or a variant Fc region. Although the boundaries of the Fc region of an immunoglobulin heavy chain might vary, the human IgG heavy chain Fc region is usually defined to stretch from an amino acid residue at position Cys226, or from Pro230, to the carboxyl-terminus thereof. The residue designations in this application are based on the EU numbering scheme of the constant domain (Edelman et al., Proc. Natl. Acad. Sci. USA, 63(1):78-85 (1969).

As used in the art, “Fc receptor” and “FcR” describe a receptor that binds to the Fc region of an antibody. The preferred FcR is a native sequence human FcR. Moreover, a preferred FcR is one which binds an IgG antibody (a gamma receptor) and includes receptors of the FcγRI, FcγRII, and FcγRIII subclasses, including allelic variants and alternatively spliced forms of these receptors. FcγRII receptors include FcγRIIA (an “activating receptor”) and FcγRIIB (an “inhibiting receptor”), which have similar amino acid sequences that differ primarily in the cytoplasmic domains thereof. FcRs are reviewed in Ravetch and Kinet, 1991, Ann. Rev. Immunol., 9:457-92; Capel et al., 1994, Immunomethods, 4:25-34; and de Haas et al., 1995, J. Lab. Clin. Med., 126:330-41. “FcR” also includes the neonatal receptor, FcRn, which is responsible for the transfer of maternal IgGs to the fetus (Guyer et al., 1976 J. Immunol., 117:587; and Kim et al., 1994, J. Immunol., 24:249).

By an antibody with an epitope that “overlaps” with another (second) epitope or with a surface on PCSK9 that interacts with the EGF-like domain of the LDLR is meant the sharing of space in terms of the PCSK9 residues that are interacted with. To calculate the percent of overlap, for example, the percent overlap of the claimed antibody's PCSK9 epitope with the surface of PCSK9 which interacts with the EGF-like domain of the LDLR, the surface area of PCSK9 buried when in complex with the LDLR is calculated on a per-residue basis. The buried area is also calculated for these residues in the PCSK9:antibody complex. To prevent more than 100% possible overlap, surface area for residues that have higher buried surface area in the PCSK9:antibody complex than in LDLR:PCSK9 complex is set to values from the LDLR:PCSK9 complex (100%). Percent surface overlap is calculated by summing over all of the LDLR:PCSK9 interacting residues and is weighted by the interaction area.

A “functional Fc region” possesses at least one effector function of a native sequence Fc region. Exemplary “effector functions” include C1q binding; complement dependent cytotoxicity; Fc receptor binding; antibody-dependent cell-mediated cytotoxicity; phagocytosis; down-regulation of cell surface receptors (e.g., B cell receptor), etc. Such effector functions generally require the Fc region to be combined with a binding domain (e.g., an antibody variable domain) and can be assessed using various assays known in the art for evaluating such antibody effector functions.

A “native sequence Fc region” comprises an amino acid sequence identical to the amino acid sequence of an Fc region found in nature. A “variant Fc region” comprises an amino acid sequence which differs from that of a native sequence Fc region by virtue of at least one amino acid modification, yet retains at least one effector function of the native sequence Fc region. Preferably, the variant Fc region has at least one amino acid substitution compared to a native sequence Fc region or to the Fc region of a parent polypeptide, e.g., from about one to about ten amino acid substitutions, and preferably, from about one to about five amino acid substitutions in a native sequence Fc region or in the Fc region of the parent polypeptide. The variant Fc region herein will preferably possess at least about 80% sequence identity with a native sequence Fc region and/or with an Fc region of a parent polypeptide, and most preferably, at least about 90% sequence identity therewith, more preferably, at least about 95%, at least about 96%, at least about 97%, at least about 98%, at least about 99% sequence identity therewith.

As used herein, the terms “atorvastatin”, “cerivastatin”, “fluvastatin”, “lovastatin”, “mevastatin”, “pitavastatin”, “pravastatin”, “rosuvastatin” and “simvastatin” include atorvastatin, cerivastatin, fluvastatin, lovastatin, mevastatin, pitavastatin, pravastatin, rosuvastatin, simvastatin, respectively, and any pharmaceutically acceptable salts, or stereoisomers, thereof. As used herein, the term “pharmaceutically acceptable salt” includes salts that are physiologically tolerated by a patient. Such salts are typically prepared from inorganic acids or bases and/or organic acids or bases. Examples of these acids and bases are well known to those of ordinary skill in the art.

As used herein, “treatment” is an approach for obtaining beneficial or desired clinical results. For purposes of this invention, beneficial or desired clinical results include, but are not limited to, one or more of the following: enhancement of LDL clearance and reducing incidence or amelioration of aberrant cholesterol and/or lipoprotein levels resulting from metabolic and/or eating disorders, or including hypercholesterolemia (e.g., HetFH or HoFH), dyslipidemia (e.g., mixed dyslipidemia), hyperlipidemia (e.g., heterozygous or homozygous familial and non-familial), atherosclerosis, ACS, and, more generally, cardiovascular disease (CVD).

“Reducing incidence” means any of reducing severity (which can include reducing need for and/or amount of (e.g., exposure to) other drugs and/or therapies generally used for this condition. As is understood by those skilled in the art, individuals may vary in terms of their response to treatment, and, as such, for example, a “method of reducing incidence” reflects administering the anti-PCSK9 antagonist antibody as described herein based on a reasonable expectation that such administration may likely cause such a reduction in incidence in that particular individual (e.g., infected with HCV).

“Ameliorating” means a lessening or improvement of one or more symptoms as compared to not administering a PCSK9 antagonist antibody. “Ameliorating” also includes shortening or reduction in duration of a symptom.

As used herein, an “effective dosage,” “therapeutically effective,” or “effective amount” of drug, compound, or pharmaceutical composition is an amount sufficient to affect any one or more beneficial or desired results. For prophylactic use, beneficial or desired results include eliminating or reducing the risk, lessening the severity, or delaying the outset of the disease, including biochemical, histological and/or behavioral symptoms of the disease, its complications and intermediate pathological phenotypes presenting during development of the disease. For therapeutic use, beneficial or desired results include clinical results such as reducing hypercholesterolemia (e.g., HetFH or HoFH) or one or more symptoms of dyslipidemia (e.g., mixed dyslipidemia), hyperlipidemia (e.g., heterozygous or homozygous familial and non-familial), atherosclerosis, cardiovascular disease, or coronary heart disease, decreasing the dose of other medications required to treat the disease, enhancing the effect of another medication, and/or delaying the progression of the disease of patients. An effective dosage can be administered in one or more administrations. For purposes of this invention, an effective dosage of drug, compound, or pharmaceutical composition is an amount sufficient to accomplish prophylactic or therapeutic treatment either directly or indirectly. As is understood in the clinical context, an effective dosage of a drug, compound, or pharmaceutical composition may or may not be achieved in conjunction with another drug, compound, or pharmaceutical composition. Thus, an “effective dosage” may be considered in the context of administering one or more therapeutic agents, and a single agent may be considered to be given in an effective amount if, in conjunction with one or more other agents, a desirable result may be or is achieved.

An “individual” or a “subject” is a mammal, more preferably, a human. Mammals also include, but are not limited to, farm animals, sport animals, pets, primates (e.g., monkeys), horses, dogs, cats, mice and rats.

As used herein, “vector” means a construct, which is capable of delivering, and, preferably, expressing, one or more gene(s) or sequence(s) of interest in a host cell. Examples of vectors include, but are not limited to, viral vectors, naked DNA or RNA expression vectors, plasmid, cosmid or phage vectors, DNA or RNA expression vectors associated with cationic condensing agents, DNA or RNA expression vectors encapsulated in liposomes, and certain eukaryotic cells, such as producer cells.

As used herein, “expression control sequence” means a nucleic acid sequence that directs transcription of a nucleic acid. An expression control sequence can be a promoter, such as a constitutive or an inducible promoter, or an enhancer. The expression control sequence is operably linked to the nucleic acid sequence to be transcribed.

As used herein, “pharmaceutically acceptable carrier” or “pharmaceutical acceptable excipient” includes any material which, when combined with an active ingredient, allows the ingredient to retain biological activity and is non-reactive with the subject's immune system. Examples include, but are not limited to, any of the standard pharmaceutical carriers such as a phosphate buffered saline solution, water, emulsions such as oil/water emulsion, and various types of wetting agents. Preferred diluents for aerosol or parenteral administration are phosphate buffered saline (PBS) or normal (0.9%) saline. Compositions comprising such carriers are formulated by well-known conventional methods (see, for example, Remington's Pharmaceutical Sciences, 18th edition, A. Gennaro, ed., Mack Publishing Co., Easton, Pa., 1990; and Remington, The Science and Practice of Pharmacy, 20th Ed., Mack Publishing, 2000).

The term “kₒₙ”, as used herein, refers to the rate constant for association of an antibody to an antigen. Specifically, the rate constants (kₒₙ and k_{off}) and equilibrium dissociation constants are measured using Fab antibody fragments (i.e., univalent) and PCSK9.

The term “k_{off}”, as used herein, refers to the rate constant for dissociation of an antibody from the antibody/antigen complex.

The term “K_{D}”, as used herein, refers to the equilibrium dissociation constant of an antibody-antigen interaction.

Reference to “about” a value or parameter herein includes (and describes) embodiments that are directed to that value or parameter per se. For example, description referring to “about X” includes description of “X.” Numeric ranges are inclusive of the numbers defining the range.

It is understood that wherever embodiments are described herein with the language “comprising,” otherwise analogous embodiments described in terms of “consisting of” and/or “consisting essentially of” are also provided.

Where aspects or embodiments of the invention are described in terms of a Markush group or other grouping of alternatives, the present invention encompasses not only the entire group listed as a whole, but each member of the group individually and all possible subgroups of the main group, but also the main group absent one or more of the group members. The present invention also envisages the explicit exclusion of one or more of any of the group members in the claimed invention.

Unless otherwise defined, all technical and scientific terms used herein have the same meaning as commonly understood by one of ordinary skill in the art to which this invention belongs. Exemplary methods and materials are described herein, although methods and materials similar or equivalent to those described herein can also be used in the practice or testing of the present invention. All publications and other references mentioned herein are incorporated by reference in their entirety. In case of conflict, the present specification, including definitions, will control. Although a number of documents are cited herein, this citation does not constitute an admission that any of these documents forms part of the common general knowledge in the art. Throughout this specification and claims, the word “comprise,” or variations such as “comprises” or “comprising” will be understood to imply the inclusion of a stated integer or group of integers but not the exclusion of any other integer or group of integers. Unless otherwise required by context, singular terms shall include pluralities and plural terms shall include the singular. The materials, methods, and examples are illustrative only and not intended to be limiting.

Antibodies of the invention can be produced using techniques well known in the art, e.g., recombinant technologies, phage display technologies, synthetic technologies or combinations of such technologies or other technologies readily known in the art (see, for example, Jayasena, S. D., Clin. Chem., 45: 1628-50, 1999 and Fellouse, F. A., et al, J. Mol. Biol., 373(4):924-40, 2007).

Published information related to anti-PCSK9 antibodies includes the following publications: PCT/162009/053990, published Mar. 18, 2010 as WO 2010/029513, U.S. patent application Ser. No. 12/558,312, published Dec. 20, 2011 as U.S. Pat. No. 8,080,243, DeVay et al., J. Biol. Chem. 288:10805-10818 (2013), each of which is herein incorporated by reference in its entirety.

Exemplary methods and materials are described herein, although methods and materials similar or equivalent to those described herein can also be used in the practice or testing of the present invention. The materials, methods, and examples are illustrative only and not intended to be limiting.

Methods for Preventing or Treating Disorders Associated with High LDL-Cholesterol

In one aspect, the invention provides a method of reducing a level of LDL-cholesterol in blood of a subject infected with hepatitis C virus (HCV), comprising administering to the subject in need thereof a therapeutically effective amount of an antagonist antibody which specifically binds to a human proprotein convertase subtilisin kexin type 9 (PCSK9) of SEQ ID NO:1.

In some embodiments, provided is an antagonist antibody which specifically binds to a human PCSK9 of SEQ ID NO:1 for use in reducing a level of LDL-cholesterol in blood of a subject infected with HCV or at high risk of contracting HCV.

In some embodiments, provided is a use of an antagonist antibody which specifically binds to a human PCSK9 of SEQ ID NO:1 in the manufacture of a medicament for reducing a level of LDL-cholesterol in blood of a subject infected with HCV or at high risk of contracting HCV.

The method or use described herein can be used in the prevention and/or treatment of conditions associated with high levels of LDL-cholesterol in a subpopulation of patients infected with HCV or at high risk of contracting HCV. In one variation, this subpopulation of HCV-infected or HCV-high risk patients is intolerant of statins or for whom statins are contraindicated.

HCV belongs to the genus of Hepacivirus in the Flaviviridae family. HCV is a leading cause of both acute and chronic hepatitis C infection, including, but not limited to, liver cirrhosis and hepatocellular carcinoma. Majority of the patients infected with HCV progress to develop these liver-associated diseases.

As used herein, “at high risk of contracting HCV” in a subject refers to those who are at risk for developing HCV infections, including, but not limited to, intravenous (IV) drug users (e.g., dirty needles (past and present)), health care workers (e.g., needle stick accidents), subjects who received blood/blood product transfusions before 1992 (e.g., when viral screening began), mother passing virus to fetus, subjects receiving long term dialysis, and subjects at risk or infected with human immunodeficiency virus (HIV).

The cholesterol and lipoprotein metabolism related disorders include, but are not limited to, hypercholesterolemia (e.g., HetFH or HoFH), dyslipidemia (e.g., mixed dyslipidemia), hyperlipidemia (e.g., heterozygous or homozygous familial and non-familial), atherosclerosis, acute coronary syndrome and, more generally, and cardiovascular disease (CVD). In some embodiments, CVD or cardiovascular events include, but are not limited to, myocardial infarction, hospitalization for heart failure (HF), hospitalization for unstable angina, stroke, cardiovascular (CV) death, and hospitalization for revascularization.

In some embodiments, the method or use comprises administering an initial dose of about 0.025 mg/kg to about 20 mg/kg of the anti-PCSK9 antagonist antibody that specifically binds to the human PCSK9 of SEQ ID NO: 1. In some embodiments, the anti-PCSK9 antagonist antibody blocks LDLR binding to the human PCSK9 of SEQ ID NO: 1. In some embodiments, the anti-PCSK9 antagonist antibody interacts with the EGF-like domain of the LDLR (e.g., SEQ ID NO: 15 or amino acid residues 314-353 of SEQ ID NO: 16). In some embodiments, the anti-PCSK9 antagonist antibody is alirocumab (PRALUENT™); evolocumab (REPATHA™); REGN728; LGT 209; RG7652; LY3015014; L1L3 (see, e.g., U.S. Pat. No. 8,080,243); 31H4, 11F1, 8A1, 8A3, or 3C4 (see, e.g., U.S. Pat. No. 8,030,457); 300N (see, e.g., U.S. Pat. No. 8,062,640); or 1D05 (see, e.g., U.S. Pat. No. 8,188,234). In some embodiments, the anti-PCSK9 antibody is bococizumab, evolocumab (REPATHA™), or alirocumab (PRALUENT™). In some embodiments, the anti-PCSK9 antagonist antibody recognizes an epitope on human PCSK9 comprising amino acid residues 153-155, 194, 195, 197, 237-239, 367, 369, 374-379 and/or 381 of the PCSK9 amino acid sequence of SEQ ID NO: 1. In some embodiments, the anti-PCSK9 antagonist antibody recognizes an epitope on human PCSK9 comprising amino acid residues 153, 154, 194, 238, 369, 374, 377, and/or 379 of the PCSK9 amino acid sequence of SEQ ID NO: 1. In some embodiments, the initial dose for the anti-PCSK9 antagonist antibody is about any of 0.025 mg/kg, 0.25 mg/kg, 0.5 mg/kg, 0.75 mg/kg, 1 mg/kg, 1.5 mg/kg, 2 mg/kg, 2.5 mg/kg, 3 mg/kg, 3.5 mg/kg, 4 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, or 20 mg/kg. In some embodiments, the maintenance dose is administered at least any of weekly, every other week, every three weeks, every four weeks, every five weeks, every six weeks, every seven weeks, every eight weeks, every nine weeks, every ten weeks, every eleven weeks, or every twelve weeks. In some embodiments, the initial dose and the first subsequent and additional subsequent doses are separated in time from each other by at least about two weeks or about four weeks.

In some embodiments, the method or use comprises administering a fixed dose of about 0.25 mg to about 2000 mg of the anti-PCSK9 antagonist antibody specifically binds to the human PCSK9 of SEQ ID NO: 1. In some embodiments, the anti-PCSK9 antagonist antibody blocks LDLR binding to the human PCSK9 of SEQ ID NO: 1. In some embodiments, the anti-PCSK9 antagonist antibody interacts with the EGF-like domain of the LDLR (e.g., SEQ ID NO: 15 or amino acid residues 314-353 of SEQ ID NO: 16). In some embodiments, the anti-PCSK9 antagonist antibody is alirocumab (PRALUENT™); evolocumab (REPATHA™); REGN728; LGT209; RG7652; LY3015014; L1L3 (see, e.g., U.S. Pat. No. 8,080,243); 31H4, 11F1, 12H11, 8A1, 8A3, or 3C4 (see, e.g., U.S. Pat. No. 8,030,457); 300N (see, e.g., U.S. Pat. No. 8,062,640); or 1D05 (see, e.g., U.S. Pat. No. 8,188,234) In some embodiments, the anti-PCSK9 antagonist antibody recognizes an epitope on human PCSK9 comprising amino acid residues 153-155, 194, 195, 197, 237-239, 367, 369, 374-379 and/or 381 of the PCSK9 amino acid sequence of SEQ ID NO: 1. In some embodiments, the anti-PCSK9 antagonist antibody recognizes an epitope on human PCSK9 comprising amino acid residues 153, 154, 194, 238, 369, 374, 377, and/or 379 of the PCSK9 amino acid sequence of SEQ ID NO: 1. In some embodiments, the fixed dose for the anti-PCSK9 antagonist antibody is about any of 0.25 mg, 0.25 mg, 0.5 mg, 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, 50 mg, 51 mg, 52 mg, 53 mg, 54 mg, 55 mg, 56 mg, 57 mg, 58 mg, 59 mg, 60 mg, 61 mg, 62 mg, 63 mg, 64 mg, 65 mg, 66 mg, 67 mg, 68 mg, 69 mg, 70 mg, 71 mg, 72 mg, 73 mg, 74 mg, 75 mg, 76 mg, 77 mg, 78 mg, 79 mg, 80 mg, 81 mg, 82 mg, 83 mg, 84 mg, 85 mg, 86 mg, 87 mg, 88 mg, 89 mg, 90 mg, 91 mg, 92 mg, 93 mg, 94 mg, 95 mg, 96 mg, 99 mg, 98 mg, 99 mg, 100 mg, 101 mg, 102 mg, 103 mg, 104 mg, 105 mg, 106 mg, 107 mg, 108 mg, 109 mg, 110 mg, 111 mg, 112 mg, 113 mg, 114 mg, 115 mg, 116 mg, 117 mg, 118 mg, 119 mg, 120 mg, 121 mg, 122 mg, 123 mg, 124 mg, 125 mg, 126 mg, 127 mg, 128 mg, 129 mg, 130 mg, 131 mg, 132 mg, 133 mg, 134 mg, 135 mg, 136 mg, 137 mg, 138 mg, 139 mg, 140 mg, 141 mg, 142 mg, 143 mg, 144 mg, 145 mg, 146 mg, 147 mg, 148 mg, 149 mg, 150 mg, 151 mg, 152 mg, 153 mg, 154 mg, 155 mg, 156 mg, 157 mg, 158 mg, 159 mg, 160 mg, 161 mg, 162 mg, 163 mg, 164 mg, 165 mg, 166 mg, 167 mg, 168 mg, 169 mg, 170 mg, 171 mg, 172 mg, 173 mg, 174 mg, 175 mg, 176 mg, 177 mg, 178 mg, 179 mg, 180 mg, 181 mg, 182 mg, 183 mg, 184 mg, 185 mg, 186 mg, 187 mg, 188 mg, 189 mg, 190 mg, 191 mg 192 mg, 193 mg, 194 mg, 195 mg, 196 mg, 199 mg, 198 mg, 199 mg, 200 mg, 250, 300, 350, 400, 450, or 500 mg, 600 mg, 700 mg, 800 mg, 900 mg, 1000 mg, 1100 mg, 1200 mg, 1300 mg, 1400 mg, 1500 mg, 1600 mg, 1700 mg, 1800 mg, 1900 mg, or 2000 mg. In some embodiments, the anti-PCSK9 antagonist antibody is administered weekly, every other week, every three weeks, every four weeks, every five weeks, every six weeks, every seven weeks, every eight weeks, every nine weeks, every ten weeks, every eleven weeks, or every twelve weeks. In some embodiments, both the anti-PCSK9 antagonist antibody is administered every two weeks or every four weeks.

The PCSK9 antagonist antibody can further be administered according to one or more dosing regimens disclosed herein to an individual on stable doses of a statin. The stable doses can be, for example without limitation, a daily dose or an every-other-day dose of a statin. A variety of statins known to those of skill in the art, and include, for example without limitation, atorvastatin, simvastatin, lovastatin, pravastatin, rosuvastatin, fluvastatin, cerivastatin, mevastatin, pitavastatin, and statin combination therapies. Non-limiting examples of statin combination therapies include atorvastatin plus amlodipine (CADUET™), simvastatin plus ezetimibe (VYTORIN™), lovastatin plus niacin (ADVICOR™), and simvastatin plus niacin (SIMCOR™).

In some embodiments, an individual has been on stable doses of a statin for at least one, two, three, four, five or six weeks prior to administration of an initial dose of the anti-PCSK9 antagonist antibody as described herein. Preferably, the individual on stable doses of a statin has a fasting LDL-C greater than or equal to about 70 mg/dL prior to administration of an initial dose of the anti-PCSK9 antagonist antibody. In some embodiments, the individual on stable doses of a statin has a fasting LDL-C greater than or equal to about 80, 90, 100, 110, 120, 130, 140, 150, 160, 170, 180, 190 or 200 mg/dL prior to administration of an initial dose of the PCSK9 antagonist antibody.

In some embodiments, an individual had been on stable doses of a statin (e.g., 1 day, 14 days, 1 month, 2 months, 3 months, 1 year, 2 years ago, etc.) prior to administration of an initial dose of the PCSK9 antagonist antibody as described herein, and initiate the statin doses with the PCSK9 antagonist antibody dosing regimen at the same time.

For the purpose of the present invention, a typical statin dose might range from about 1 mg to about 80 mg, depending on the factors mentioned above. For example, a statin dose of about any of 0.3 mg, 0.5 mg, 1 mg, 2.5 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 30 mg, 31 mg, 32 mg, 33 mg, 34 mg, 35 mg, about 36 mg, about 37 mg, about 38 mg, 39 mg, 40 mg, 41 mg, 42 mg, 43 mg, 44 mg, 45 mg, 46 mg, 47 mg, 48 mg, 49 mg, 50 mg, 51 mg, 52 mg, 53 mg, 54 mg, 55 mg, about 56 mg, about 57 mg, about 58 mg, 59 mg, 60 mg, 61 mg, 62 mg, 63 mg, 64 mg, 65 mg, 66 mg, 67 mg, 68 mg, 69 mg, 70 mg, 71 mg, 72 mg, 73 mg, 74 mg, 75 mg, 76 mg, 77 mg, 78 mg, 79 mg, or 80 mg may be used.

In preferred embodiments, a dose of 40 mg or 80 mg atorvastatin is used. In other embodiments, a dose of 20 mg or 40 mg rosuvastatin is used. In other embodiments, a dose of 40 mg or 80 mg simvastatin is used.

In some embodiments, the PCSK9 antagonist antibody can also be administered according to one or more dosing regimens disclosed herein to an individual on HCV medication. For example, a subject has been on the HCV medication of interferon alpha (e.g., pegylated interferon alpha 2a or 2b; non-pegylated interferon), ribavirin, boceprevir, telaprevir, sofosbuvir, ledipasvir, and/or simeprevir, and/or any combination of thereof (e.g., for at least 1 week) prior to administration of an initial dose of the anti-PCSK9 antagonist antibody as described herein. In some embodiments, the initial dose of the anti-PCSK9 antagonist antibody can also be administered before a subject has been on the HCV medication as described herein (e.g., combination antiviral therapy with interferon alpha and ribavirin etc.).

Advantageously, administration of the anti-PCSK9 antagonist antibody results in lower blood LDL-cholesterol in a subpopulation of subjects injected with HCV or at high risk of contracting HCV. Preferably, blood LDL-cholesterol is at least about 10% or 15% lower than before administration. More preferably, blood LDL-cholesterol is at least about 20% lower than before administration of the antibody. Yet more preferably, blood LDL-cholesterol is at least 30% lower than before administration of the antibody. Advantageously, blood LDL-cholesterol is at least 40% lower than before administration of the antibody. More advantageously, blood LDL-cholesterol is at least 50% lower than before administration of the antibody. Very preferably, blood LDL-cholesterol is at least 60% lower than before administration of the antibody. Most preferably, blood LDL-cholesterol is at least 70% lower than before administration of the antibody.

The anti-PCSK9 antagonist antibody described herein can be administered to a subject via any suitable route. It should be apparent to a person skilled in the art that the examples described herein are not intended to be limiting but to be illustrative of the techniques available. Accordingly, in some embodiments, the anti-PCSK9 antagonist antibody is administered to an individual in accord with known methods, such as intravenous administration, e.g., as a bolus or by continuous infusion over a period of time, by subcutaneous, intramuscular, intraperitoneal, intracerebrospinal, transdermal, intra-articular, sublingually, intrasynovial, via insufflation, intrathecal, oral, inhalation or topical routes. Administration can be systemic, e.g., intravenous administration, or localized. Commercially available nebulizers for liquid formulations, including jet nebulizers and ultrasonic nebulizers are useful for administration. Liquid formulations can be directly nebulized and lyophilized powder can be nebulized after reconstitution.

Alternatively, the anti-PCSK9 antagonist antibody can be aerosolized using a fluorocarbon formulation and a metered dose inhaler, or inhaled as a lyophilized and milled powder.

In some embodiments, the anti-PCSK9 antagonist antibody is administered via site-specific or targeted local delivery techniques. Examples of site-specific or targeted local delivery techniques include various implantable depot sources of the anti-PCSK9 antagonist antibody or local delivery catheters, such as infusion catheters, indwelling catheters, or needle catheters, synthetic grafts, adventitial wraps, shunts and stents or other implantable devices, site specific carriers, direct injection, or direct application. See, e.g., PCT Publ. No. WO 00/53211 and U.S. Pat. No. 5,981,568.

With respect to all methods described herein, reference to any anti-PCSK9 antagonist antibody also includes compositions comprising one or more additional agents. These compositions may further comprise suitable excipients, such as pharmaceutically acceptable excipients including buffers, which are well known in the art. The present invention can be used alone or in combination with other conventional methods of treatment.

Various formulations of an anti-PCSK9 antagonist antibody may be used for combination administration. In some embodiments, the anti-PCSK9 antagonist antibody can be administered neat. In some embodiments, the anti-PCSK9 antagonist antibody can also be administered via inhalation. In some embodiments, the anti-PCSK9 antagonist antibody and a pharmaceutically acceptable excipient may be in various formulations. Pharmaceutically acceptable excipients are known in the art, and are relatively inert substances that facilitate administration of a pharmacologically effective substance. For example, an excipient can give form or consistency, or act as a diluent. Suitable excipients include but are not limited to stabilizing agents, wetting and emulsifying agents, salts for varying osmolarity, encapsulating agents, buffers, and skin penetration enhancers. Excipients as well as formulations for parenteral and nonparenteral drug delivery are set forth in Remington, The Science and Practice of Pharmacy, 21st Ed., Mack Publishing (2005).

These agents can be combined with pharmaceutically acceptable vehicles such as saline, Ringer's solution, dextrose solution, and the like. The particular dosage regimen, i.e., dose, timing and repetition, will depend on the particular individual and that individual's medical history.

Acceptable carriers, excipients, or stabilizers are nontoxic to recipients at the dosages and concentrations employed, and may comprise buffers such as phosphate, citrate, and other organic acids; salts such as sodium chloride; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens, such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

Liposomes containing the anti-PCSK9 antagonist antibody are prepared by methods known in the art, such as described in Epstein, et al., 1985, Proc. Natl. Acad. Sci. USA 82:3688; Hwang, et al., 1980, Proc. Natl Acad. Sci. USA 77:4030; and U.S. Pat. Nos. 4,485,045 and 4,544,545. Liposomes with enhanced circulation time are disclosed in U.S. Pat. No. 5,013,556. Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter.

The active ingredients may also be entrapped in microcapsules prepared, for example, by coacervation techniques or by interfacial polymerization, for example, hydroxymethylcellulose or gelatin-microcapsules and poly-(methylmethacrylate) microcapsules, respectively, in colloidal drug delivery systems (for example, liposomes, albumin microspheres, microemulsions, nano-particles and nanocapsules) or in macroemulsions. Such techniques are disclosed in Remington, The Science and Practice of Pharmacy, 21st Ed., Mack Publishing (2005).

Sustained-release preparations may be prepared. Suitable examples of sustained-release preparations include semipermeable matrices of solid hydrophobic polymers containing the antibody, which matrices are in the form of shaped articles, e.g., films, or microcapsules. Examples of sustained-release matrices include polyesters, hydrogels (for example, poly(2-hydroxyethyl-methacrylate), or ‘poly(vinylalcohol)), polylactides (U.S. Pat. No. 3,773,919), copolymers of L-glutamic acid and 7 ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), sucrose acetate isobutyrate, and poly-D-(−)-3-hydroxybutyric acid.

The formulations to be used for in vivo administration must be sterile. This is readily accomplished by, for example, filtration through sterile filtration membranes. Therapeutic anti-PCSK9 antagonist antibodies are generally placed into a container having a sterile access port, for example, an intravenous solution bag or vial having a stopper pierceable by a hypodermic injection needle.

Suitable emulsions may be prepared using commercially available fat emulsions, such as Intralipid™, Liposyn™, Infonutrol™, Lipofundin™ and Lipiphysan™. The active ingredient may be either dissolved in a pre-mixed emulsion composition or alternatively it may be dissolved in an oil (e.g., soybean oil, safflower oil, cottonseed oil, sesame oil, corn oil or almond oil) and an emulsion formed upon mixing with a phospholipid (e.g., egg phospholipids, soybean phospholipids or soybean lecithin) and water. It will be appreciated that other ingredients may be added, for example glycerol or glucose, to adjust the tonicity of the emulsion. Suitable emulsions typically contain up to 20% oil, for example, between 5 and 20%. The fat emulsion can comprise fat droplets between 0.1 and 1.0 μm, particularly 0.1 and 0.5 μm, and have a pH in the range of 5.5 to 8.0.

The emulsion compositions can be those prepared by mixing an anti-PCSK9 antagonist antibody with Intralipid™ or the components thereof (soybean oil, egg phospholipids, glycerol and water).

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as set out above. In some embodiments, the compositions are administered by the oral or nasal respiratory route for local or systemic effect. Compositions in preferably sterile pharmaceutically acceptable solvents may be nebulized by use of gases. Nebulized solutions may be breathed directly from the nebulizing device or the nebulizing device may be attached to a face mask, tent or intermittent positive pressure breathing machine. Solution, suspension or powder compositions may be administered, preferably orally or nasally, from devices which deliver the formulation in an appropriate manner.

### PCSK9 Antagonist Antibodies

A description follows as to an exemplary technique for the production of the antibodies used in accordance with the present invention. The PCSK9 antigen to be used for production of antibodies may be, e.g. full-length human PCSK9, full length mouse PCSK9, and various peptides fragments of PCSK9. Other forms of PCSK9 useful for generating antibodies will be apparent to those skilled in the art.

As will be appreciated, antibodies for use in the present invention may be derived from hybridomas but can also be expressed in cell lines other than hybridomas. Sequences encoding the cDNAs or genomic clones for the particular antibodies can be used for transformation of suitable mammalian or nonmammalian host cells. Mammalian cell lines available as hosts for expression are well known in the art and include many immortalized cell lines available from the American Type Culture Collection (ATCC), including but not limited to Chinese hamster ovary (CHO) cells, NSO, HeLa cells, baby hamster kidney (BHK) cells, monkey kidney cells (COS), and human hepatocellular carcinoma cells (e.g., Hep G6). Non-mammalian cells can also be employed, including bacterial, yeast, insect, and plant cells. Site directed mutagenesis of the antibody CH6 domain to eliminate glycosylation may be preferred in order to prevent changes in either the immunogenicity, pharmacokinetic, and/or effector functions resulting from non-human glycosylation. The glutamine synthase system of expression is discussed in whole or part in connection with European Patents 616 846, 656 055, and 363 997 and European Patent Application 89303964.4. Further, a dihydrofolate reductase (DHFR) expression system, including those known in the art, can be used to produce the antibody.

In some embodiments, the invention is practiced using the anti-PCSK9 antagonist antibody blocking the LDLR binding of the human PCSK9 (e.g., SEQ ID NO: 1). In some embodiments, the anti-PCSK9 antagonist antibody interacts with the EGF-like domain of the LDLR (e.g., SEQ ID NO: 15 or amino acid residues 314-353 of SEQ ID NO: 16). In some embodiments, the anti-PCSK9 antagonist antibody is alirocumab (PRALUENT™); evolocumab (REPATHA™); REGN728; LGT209; RG7652; LY3015014; L1L3 (see, e.g., U.S. Pat. No. 8,080,243); 31H4, 11F1, 12H11, 8A1, 8A3, or 3C4 (see, e.g., U.S. Pat. No. 8,030,457); 300N (see, e.g., U.S. Pat. No. 8,062,640); or 1D05 (see, e.g., U.S. Pat. No. 8,188,234). In some embodiments, the anti-PCSK9 antibody is bococizumab, evolocumab (REPATHA™), or alirocumab (PRALUENT™).

In some embodiments, the invention is practiced using the anti-PCSK9 antagonist antibody recognizing an epitope of human PCSK9 comprising amino acid residues 153-155, 194, 195, 197, 237-239, 367, 369, 374-379 and/or 381 of the human PCSK9 (e.g., SEQ ID NO: 1).

In some embodiments, the invention is practiced using the anti-PCSK9 antagonist antibody recognizing an epitope of human PCSK9 comprising amino acid residues 153, 154, 194, 238, 369, 374, 377, and/or 379 of the human PCSK9 (e.g., SEQ ID NO: 1).

In some embodiments, the invention is practiced using an antibody comprising three CDRS from a heavy chain variable region having the amino acid sequence shown in SEQ ID NO: 2 and three CDRS from a light chain variable region having the amino acid sequence shown in SEQ ID NO: 3.

In some embodiments, the invention is practiced using an antibody that specifically binds PCSK9 comprising a VH complementary determining region one (CDR1) having the amino acid sequence shown in SEQ ID NO: 4 (SYYMH), SEQ ID NO: 5 (GYTFTSY), or SEQ ID NO: 6 (GYTFTSYYMH); a VH CDR2 having the amino acid sequence shown in SEQ ID NO: 7 (EISPFGGRTNYNEKFKS) or SEQ ID NO: 8 (SPFGGR), and/or VH CDR3 having the amino acid sequence shown in SEQ ID NO: 9 (ERPLYASDL), or a variant thereof having one or more conservative amino acid substitutions in said sequences of CDR1, CDR2, and/or CDR3, wherein the variant retains essentially the same binding specificity as the CDR defined by said sequences. Preferably, the variant comprises up to about ten amino acid substitutions and, more preferably, up to about four amino acid substitutions.

In some embodiments, the invention is practiced using an antibody comprising a VL CDR1 having the amino acid sequence shown in SEQ ID NO: 10 (RASQGISSALA), a CDR2 having the amino acid sequence shown in SEQ ID NO: 11 (SASYRYT), and/or CDR3 having the amino acid sequence shown in SEQ ID NO: 12 (QQRYSLWRT), or a variant thereof having one or more conservative amino acid substitutions in said sequences of CDR1, CDR2, and/or CDR3, wherein the variant retains essentially the same binding specificity as the CDR1 defined by said sequences. Preferably, the variant comprises up to about ten amino acid substitutions and, more preferably, up to about four amino acid substitutions.

In some embodiments, the invention is practiced using an antibody having a heavy chain sequence comprising or consisting of SEQ ID NO: 14, with or without the C-terminal lysine of SEQ ID NO: 14, and a light chain sequence comprising or consisting of SEQ ID NO: 13.

In some embodiments, the invention is practiced using an antibody having a heavy chain variable region comprising or consisting of the amino acid sequence shown in SEQ ID NO: 11 and a light chain variable region comprising or consisting of the amino acid sequence shown in SEQ ID NO: 12.

In some embodiments, the invention is practiced using an antibody that recognizes a first epitope of PCSK9 that is the same as or overlaps with a second epitope that is recognized by a monoclonal antibody selected from the group consisting of 5A10, which is produced by a hybridoma cell line deposited with the American Type Culture Collection and assigned accession number PTA-8986; 4A5, which is produced by a hybridoma cell line deposited with the American Type Culture Collection and assigned accession number PTA-8985; 6F6, which is produced by a hybridoma cell line deposited with the American Type Culture Collection and assigned accession number PTA-8984, and 7D4, which is produced by a hybridoma cell line deposited with the American Type Culture Collection and assigned accession number PTA-8983. In preferred embodiments, the invention is practiced using the PCSK9 antagonist antibody L1 L3 (see, PCT/162009/053990, published Mar. 18, 2010 as WO 2010/029513, and U.S. patent application Ser. No. 12/558,312, published Mar. 18, 2010 as US 2010/0068199).

In some embodiments, a variant of the anti-PCSK9 antagonist antibody as described herein comprises up to about twenty amino acid substitutions and more preferably, up to about eight amino acid substitutions. Preferably, the antibody further comprises an immunologically inert constant region, and/or the antibody has an isotype that is selected from the group consisting of IgG₂, IgG₄, IgG_{2Δa}, IgG_{4Δc}, IgG₄ S228P, IgG_{4Δb} S228P and IgG_{4Δc} S228P. In another preferred embodiment, the constant region is aglycosylated Fc.

The antibodies useful in the present invention can encompass monoclonal antibodies, polyclonal antibodies, antibody fragments (e.g., Fab, Fab′, F(ab′)2, Fv, Fc, etc.), chimeric antibodies, bispecific antibodies, heteroconjugate antibodies, single chain (ScFv), mutants thereof, fusion proteins comprising an antibody portion (e.g., a domain antibody), human antibodies, humanized antibodies, and any other modified configuration of the immunoglobulin molecule that comprises an antigen recognition site of the required specificity, including glycosylation variants of antibodies, amino acid sequence variants of antibodies, and covalently modified antibodies. The antibodies may be murine, rat, human, or any other origin (including chimeric or humanized antibodies).

In some embodiments, the PCSK9 antagonist antibody is a monoclonal antibody. The PCSK9 antagonist antibody can also be humanized. In other embodiments, the antibody is human.

In some embodiments, the antibody comprises a modified constant region, such as a constant region that is immunologically inert, that is, having a reduced potential for provoking an immune response. In some embodiments, the constant region is modified as described in Eur. J. Immunol., 1999, 29:2613-2624; PCT Publ. No. WO99/58572; and/or UK Patent Application No. 9809951.8. The Fc can be human IgG₂ or human IgG₄. The Fc can be human IgG₂ containing the mutation A330P331 to S330S331 (IgG_{2Δa}), in which the amino acid residues are numbered with reference to the wild type IgG2 sequence. Eur. J. Immunol., 1999, 29:2613-2624. In some embodiments, the antibody comprises a constant region of IgG₄ comprising the following mutations (Armour et al., 2003, Molecular Immunology 40 585-593): E233F234L235 to P233V234A235 (IgG_{4Δc}), in which the numbering is with reference to wild type IgG4. In yet another embodiment, the Fc is human IgG₄ E233F234L235 to P233V234A235 with deletion G236 (IgG_{4Δb}). In another embodiment the Fc is any human IgG₄ Fc (IgG₄, IgG_{4Δb} or IgG_{4Δc}) containing hinge stabilizing mutation S228 to P228 (Aalberse et al., 2002, Immunology 105, 9-19). In another embodiment, the Fc can be aglycosylated Fc.

In some embodiments, the constant region is aglycosylated by mutating the oligosaccharide attachment residue (such as Asn297) and/or flanking residues that are part of the glycosylation recognition sequence in the constant region. In some embodiments, the constant region is aglycosylated for N-linked glycosylation enzymatically. The constant region may be aglycosylated for N-linked glycosylation enzymatically or by expression in a glycosylation deficient host cell.

The anti-PCSK9 antagonist antibody as described herein can also be used in conjunction with other PCSK9 antagonists or PCSK9 receptor antagonists. For example, one or more of the following PCSK9 antagonists may be used: an antisense molecule directed to a PCSK9 (including an anti-sense molecule directed to a nucleic acid encoding PCSK9), a PCSK9 inhibitory compound, and a PCSK9 structural analog. A PCSK9 antagonist antibody can also be used in conjunction with other agents that serve to enhance and/or complement the effectiveness of the agents.

### Kits

The invention also provides kits or an article of manufacture comprising an anti-PCSK9 antagonist antibody and instructions for use. Accordingly, in some embodiments, provided is a kit or an article of manufacture, comprising a container, a composition within the container comprising a PCSK9 antagonist antibody, and a package insert containing instructions to administer a therapeutically effective amount of the PCSK9 antagonist antibody for reducing a level of LDL-cholesterol in blood of a subject infected with HCV or at high risk of contracting HCV.

Kits of the invention include one or more containers comprising an anti-PCSK9 antagonist antibody described herein and instructions for use in accordance with the methods of the invention described herein. Generally, these instructions comprise a description of administration of the anti-PCSK9 antagonist antibody for the above described therapeutic treatments. In some embodiments, kits are provided for producing a single-dose administration unit. In certain embodiments, the kit can contain both a first container having a dried protein and a second container having an aqueous formulation. In certain embodiments, kits containing single and multi-chambered pre-filled syringes (e.g., liquid syringes and lyosyringes) are included.

The instructions relating to the use of an anti-PCSK9 antagonist antibody generally include information as to dosage, dosing schedule, and route of administration for the intended treatment. The containers may be unit doses, bulk packages (e.g., multi-dose packages) or sub-unit doses. Instructions supplied in the kits of the invention are typically written instructions on a label or package insert (e.g., a paper sheet included in the kit), but machine-readable instructions (e.g., instructions carried on a magnetic or optical storage disk) are also acceptable.

The kits of this invention are in suitable packaging. Suitable packaging includes, but is not limited to, vials, bottles, jars, flexible packaging (e.g., sealed Mylar or plastic bags), and the like. Also contemplated are packages for use in combination with a specific device, such as an inhaler, nasal administration device (e.g., an atomizer) or an infusion device such as a minipump. A kit may have a sterile access port (for example the container may be an intravenous solution bag or a vial having a stopper pierceable by a hypodermic injection needle). The container may also have a sterile access port (for example the container may be an intravenous solution bag or a vial having a stopper pierceable by a hypodermic injection needle). At least one active agent in the composition is an anti-PCSK9 antagonist antibody. The container may further comprise a second pharmaceutically active agent.

Kits may optionally provide additional components such as buffers and interpretive information. Normally, the kit comprises a container and a label or package insert(s) on or associated with the container.

The following examples are offered for illustrative purposes only, and are not intended to limit the scope of the present invention in any way. Indeed, various modifications of the invention in addition to those shown and described herein will become apparent to those skilled in the art from the foregoing description and fall within the scope of the appended claims.

### EXAMPLES

The present invention is illustrated in further details by the following non-limiting examples. It is understood that other embodiments may be practiced given the general description provided here.

### Example 1—Anti-PCSK9 Antibody 5A10 has No Effect on Liver CD81 Expression

This example illustrates that PCSK9 antagonism does not affect CD81 expression levels in mouse liver.

Six C57B16 male mice of 12 weeks of age were randomly assigned to two groups of three mice each (n=3). Each group received 20 mg/kg of 5A10 (a mouse precursor for L1L3) or an isotype control antibody via IV dosing. At 7 days post treatment, mice were sacrificed and an approximately 0.2 g piece of liver from left lobe was harvested from each animal and snap-froze for determination of CD81 expression levels.

The harvested liver tissues were grounded and lysed in lysis buffer on ice. BCA assay kit was used to measure the total liver protein concentration. Western blot was used to determine CD81 protein expression level from the harvested liver tissue samples. Total of 0.04 mg protein per well was loaded on NuPAGE® Novex 4-12% Bis-Tris Gel (Life Technologies, Grand Island, N.Y.) and was run at 200 Volt for 40 minutes. Proteins were transferred to Nitrocellulose membrane using iBlot® Gel Transfer stacks (Life Technologies). The membrane was then blocked in Odyssey® Blocking Buffer (LI-COR, P/N 927-40000) for 1 hour at room temperature. Primary antibodies of anti-CD81 (rabbit anti-PCSK9; Santa Cruz Biotechnologies, Santa Cruz, Calif.), anti-LDLR (goat anti-Human; R&D Systems, Minneapolis, Minn.), or anti-beta-actin were diluted at 1:1000, 1:2000, and 1:2000 respectively in Odyssey Blocking Buffer. Subsequently, transferred membranes were incubated with primary antibodies for overnight at 4° C. (anti-CD81) or room temperature for 1 hr (anti-LDLR and anti-beta-actin) with gentle shaking. After incubation with different primary antibodies, the membranes were washed and incubated with fluorescently-labeled secondary antibodies (goat anti-mouse IRDye® 800 CW diluted at 1:10,000 in Odyssey Blocking Buffer) for 1 hr at room temperature with gentle shaking. At last, the membranes were washed and scanned at the 800 nm channel with Odyssey Imager (LI-COR). The signal intensity of each band was quantified using Odyssey Imager as well.

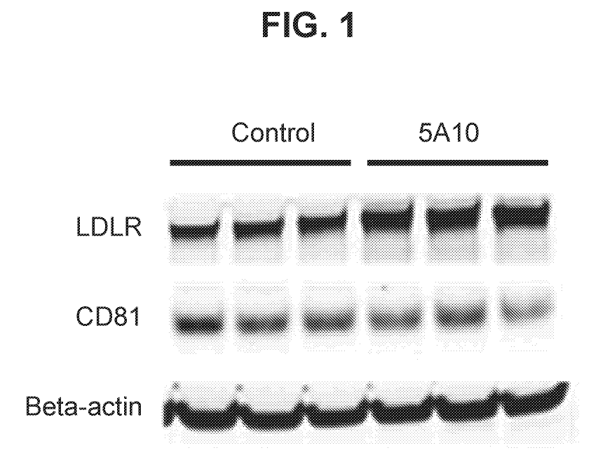

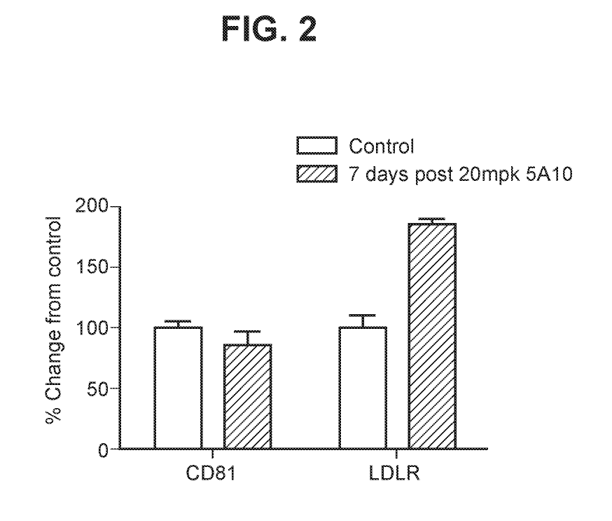

As demonstrated in FIG. 1, in mice treated with 5A10, LDLR protein levels in livers as detected by Western were induced by approximately 2 fold when compared to control mice liver samples; whereas CD81 levels were not significantly modified in these animals. FIG. 2 illustrated the quantity of each group's Western blot band signal intensity with a bar graph. In conclusion, mice liver CD81 protein levels were not affected by PCSK9 antagonism through antibody treatment.

### Example 2—PCSK9 Mediated CD81 Degradation

This example illustrates that PCSK9 mediates surface exposed CD81 degradation in a manner similar to LDLR and that PCSK9 antagonism does not affect CD81 expression levels.

HepG2 cells were cultured in DMEM supplemented with 10% FBS, L-glutamine, and pen-strep. 24 hours prior to the treatment, HepG2 cells were plated onto glass coverslips or 12-well tissue culture plates. PCSK9 in 300 nM was then added to HepG2 cells followed by 300 nM IC or 300 nM L1L3 treatment. After 6 hours, cells were processed for immunofluoresence analysis.

Treated cells were fixed with 4% formaldehyde, permeabilized, and blocked with 2 mg/ml BSA, 10% donkey serum, and 300 nM glycine in PBS. After blocking, coverslips were incubated overnight at 4 degrees with 3 μg/ml mouse anti-CD81 (BioRad) or goat anti-LDLR (R&D Systems). After washing, coverslips were incubated with 2 μg/ml donkey anti-mouse 647, or donkey anti-goat 546 secondary antibodies (Life Technologies) for 1 hour and mounted using Prolong gold with DAPI (Life Technologies).

A Leica SP3 laser scanning confocal microscope (Leica, Buffalo Grove, Ill.) was used to capture images of treated cells. Microscopy images from z stacks with 0.5 μm increments were collected using a 63×, 1.4NA objective lens at room temperature on the confocal microscope. All images shown are projections of optical sections. Data analysis was performed using Leica LAS AF software. Internalization was quantified as intensity of fluorescence signal per cell, from at least 45 (or 50) cells.

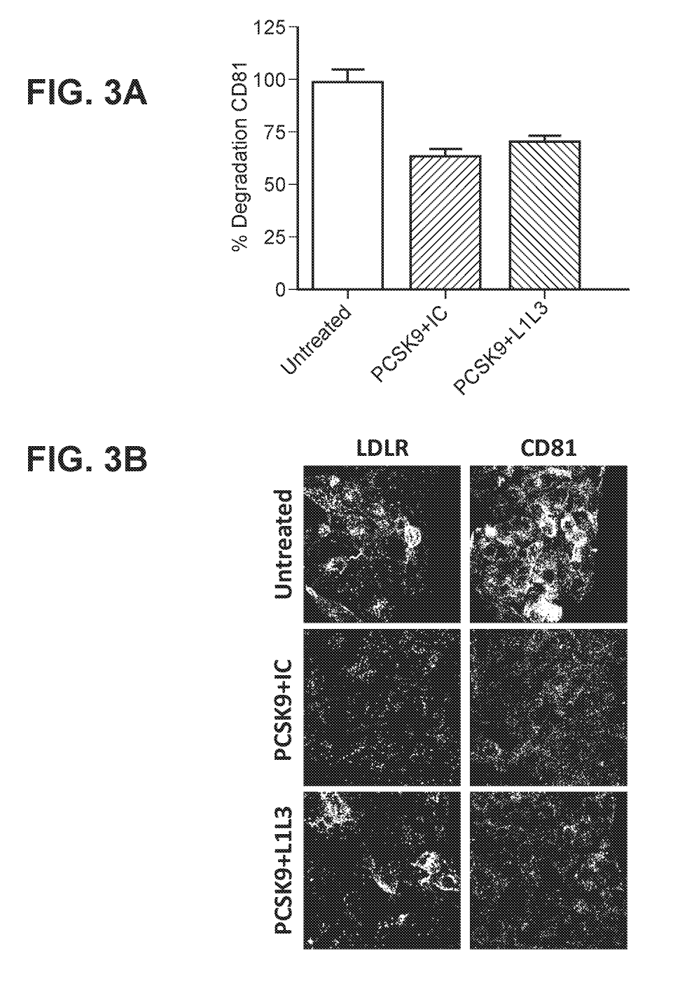

FIGS. 3A and 3B show that PCSK9 treated cells had significantly lower CD81 levels than untreated cells, indicating that PCSK9 modulates CD81 levels, albeit less effectively than LDLR. Moreover, L1L3 does not block this function of PCSK9, as L1L3 bound PCSK9 had the same effect on surface CD81 as PCSK9 combined with isotype control.

### Example 3—PCSK9 Dose Dependent Loss of CD81 and LDLR

This example illustrates that the degradation of CD81 and LDLR is PCSK9 dose dependent and that PCSK9 antagonism does not restore PCSK9 mediated CD81 degradation.

HepG2 cells were treated with different concentration of PCSK9 (e.g., 3.1 nM, 6.3 nM, 12.5 nM, 25 nM, 50 nM, 100 nM, 200 nM) or PCSK9 together with L1L3, or PCSK9 together with isotype control antibody for hours. After 6 hours, cells were collected for western blot analyses.

For western blot, lysates were first harvested and run onto a 4-12% Bis-Tris gel, and transferred to nitrocellulose membranes. Nitrocellulose membranes were blocked with Odyssey blocking buffer, incubated with primary antibodies (LDLR, TFNR (mouse anti-Human; Life Technologies), or CD81 for at least 1 hour, washed, and incubated with secondary antibodies (donkey anti-mouse 680, donkey anti-goat 800, goat anti-rabbit 680, or goat anti-mouse 800 (Licor)) before imaging on Licor Odyssey imaging system. Integrated intensity signals were measured using Odyssey software and normalized against transferrin receptor (TFNR; as indicated in figure legends).

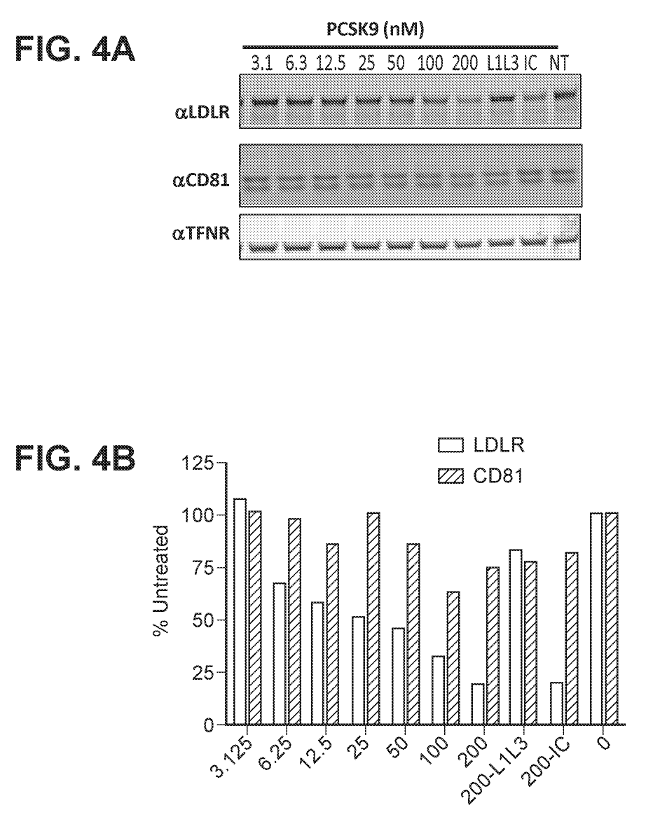

The Western blot analysis in FIG. 4 illustrated a dose dependent loss of CD81 and LDLR in the presence of PCSK9. Anti-PCSK9 antibody L1L3 restored LDLR levels but did not restore PCSK9 mediated CD81 degradation. Further, there was approximately 25% degradation of total CD81 at the highest PCSK9 concentrations, both in the presence and absence of L1L3 or IC. Together, this example indicates that PCSK9 degrades CD81 via a unique epitope that is not inhibited by L1L3 binding.

### Example 4: Pharmacokinetics and Pharmacodynamics Following Multiple Doses of L1L3 in Combination with Statin

This example illustrates a clinical trial study to evaluate LDL-C levels following multiple subcutaneous doses of PCSK9 antagonist antibody (L1L3) in human subjects with HCV infection on a statin.

This study is a randomized, multi center, double blind, placebo control, parallel group, dose-ranging study designed trial to assess the efficacy, safety and tolerability of L1L3 following monthly and twice monthly subcutaneous dosing for six months in hypercholesterolemic subjects on a statin. A total of 14 dose groups (7 without HCV infection; 7 with HCV infection) in two dosing schedules (Q28d or Q14d), with 50 subjects per dose group are planned. Protocol design is set forth in Table 1.

TABLE 1

| | Assigned Interventions (with |
|---|---|
| Arms | or without HCV infection) |
| Experimental: Q28d Dosing Arm | Group 1: Placebo, Q28d |
| Q28d dose groups will receive | Group 2: L1L3 200 mg, Q28d |
| subcutaneous administration of L1L3 | Group 3: L1L3 300 mg, Q28d |
| antibody or Placebo once a month. | |
| Experimental: Q14d Dosing Arm | Group 4: Placebo, Q14d |
| Q14d dose groups will receive | Group 5: L1L3 50 mg, Q14d |
| subcutaneous administration of L1L3 | Group 6: L1L3 100 mg, Q14d |
| antibody or Placebo every 2 weeks. | Group 7: L1L3 150 mg, Q14d |

Eligibility: ages 18 years or older.

Inclusion criteria: subjects should be receiving stable doses (at least 6 weeks) of any statin and continue on same dose of statin for the duration of this trial. Lipids should meet the following criteria on a background treatment with a statin at 2 screening visits that occur at screening and at least 7 days prior to randomization on Day 1: fasting LDL-C greater than or equal to 80 mg/dL (2.31 mmol/L); fasting TG less than or equal to 400 mg/dL (4.52 mmol/L); subject's fasting LDL-C must be greater than or equal to 80 mg/dL (2.31 mmol/L at the initial screen visit, and the value at the second visit within 7 days of randomization must be not lower than 20% of this initial value to meet eligibility criteria for this trial.

The primary outcome measure is the absolute change from baseline in LDL-C at the end of week 12 following randomization. Secondary outcome measures include the following: LDL-C is assessed as % change from baseline at the end of week 12 following randomization; steady-state L1L3 pharmacokinetic parameters derived from plasma concentrations; proportion of subjects having LDL-C less than specified limits (<100 mg/dL, <70 mg/dL, <40 mg/dL, <25 mg/dL); total cholesterol is assessed as change and % change from baseline at the end of week 12 following randomization; ApoB is assessed as change and % change from baseline at the end of week 12 following randomization; ApoA1 is assessed as change and % change from baseline at the end of week 12 following randomization; lipoprotein (a) is assessed as change and % change from baseline at the end of week 12 following randomization; HDL-cholesterol is assessed as change and % change from baseline at the end of week 12 following randomization; very low density lipoprotein-cholesterol is assessed as change and % change from baseline at the end of week 12 following randomization; triglycerides is assessed as change and % change from baseline at the end of week 12 following randomization; and non-HDL-cholesterol is assessed as change and % change from baseline at the end of week 12 following randomization.

Although the disclosed teachings have been described with reference to various applications, methods, kits, and compositions, it will be appreciated that various changes and modifications can be made without departing from the teachings herein and the claimed invention below. The foregoing examples are provided to better illustrate the disclosed teachings and are not intended to limit the scope of the teachings presented herein. While the present teachings have been described in terms of these exemplary embodiments, the skilled artisan will readily understand that numerous variations and modifications of these exemplary embodiments are possible without undue experimentation. All such variations and modifications are within the scope of the current teachings.

All references cited herein, including patents, patent applications, papers, text books, and the like, and the references cited therein, to the extent that they are not already, are hereby incorporated by reference in their entirety. In the event that one or more of the incorporated literature and similar materials differs from or contradicts this application, including but not limited to defined terms, term usage, described techniques, or the like, this application controls.

The foregoing description and Examples detail certain specific embodiments of the invention and describes the best mode contemplated by the inventors. It will be appreciated, however, that no matter how detailed the foregoing may appear in text, the invention may be practiced in many ways and the invention should be construed in accordance with the appended claims and any equivalents thereof.

It is claimed:

1. A method of reducing a level of LDL-cholesterol in blood of a subject infected with hepatitis C virus (HCV) or at high risk of contracting HCV, comprising administering to the subject in need thereof a therapeutically effective amount of an antagonist antibody which specifically binds to a human proprotein convertase subtilisin kexin type 9 (PCSK9) of SEQ ID NO:1.

2. The method of claim 1, wherein the antibody blocks LDLR binding to the PCSK9 of SEQ ID NO: 1.

3. The method of claim 1, wherein the antibody is alirocumab (PRALUENT™), evolocumab (REPATHA™), REGN728, LGT209, RG7652, LY3015014, L1L3, 31H4, 11F1, 12H11, 300N, 8A1, 8A3, 3C4, or 1D05.

4. The method of claim 1, wherein the antibody comprises a heavy chain variable region (VH) comprising complementarity determining region one (CDR1), CDR2, and CDR3 of the amino acid sequence shown in SEQ ID NO: 2; and a light chain variable region (VL) comprising CDR1, CDR2, and CDR3 of the amino acid sequence shown in SEQ ID NO: 3.

5. The method of claim 1, wherein the antibody comprises a VH CDR1 having the amino acid sequence shown in SEQ ID NO: 4, 5, or 6, a VH CDR2 having the amino acid sequence shown in SEQ ID NO:7 or 8, a VH CDR3 having the amino acid sequence shown in SEQ ID NO: 9, or a variant thereof having one or more conservative amino acid substitutions in CDR1, CDR2, and/or CDR3; and a VL CDR1 having the amino acid sequence shown in SEQ ID NO:10, a VL CDR2 having the amino acid sequence shown in SEQ ID NO:11, and a VL CDR3 having the amino acid sequence shown in SEQ ID NO: 12, or a variant thereof having one or more conservative amino acid substitutions in CDR1, CDR2, and/or CDR3.

6. The method of claim 1, wherein the antibody comprises a light chain having SEQ ID NO: 13 and a heavy chain having SEQ ID NO: 14, with or without the C-terminal lysine of SEQ ID NO: 14.

7. The method of claim 1, wherein the antibody is a full antagonist of the PCSK9-mediated effect LDL receptor (LDLR) levels as measured in vitro using an LDLR down-regulation assay in Huh7 cells.

8. The method of claim 1, further comprising administering a statin.

9. The method of claim 1, wherein the subject suffers from dyslipidemia, hyperlipidemia, hypercholesterolemia, atherosclerosis, cardiovascular disease, and/or coronary heart disease.

10. The method of claim 1, wherein the antibody is administered intravenously or subcutaneously.

11. The method of claim 1, wherein the antibody is administered at least every four weeks or every 2 weeks to the subject.

12. The method of claim 1, wherein the method comprises administering about 10 mg to about 2000 mg of the antibody to the subject.

13. The method of claim 1, wherein a statin has been administered prior to the initial dose of the antibody.

14. The method of claim 13, wherein a daily dose of a statin is administered.

15. The method of claim 13, wherein stable doses of the statin have been administered for at least about two, three, four, five, or six weeks prior to the initial dose of the antibody.

16. The method of claim 13, wherein the statin is atorvastatin, cerivastatin, fluvastatin, lovastatin, mevastatin, pitavastatin, pravastatin, rosuvastatin, simvastatin, or any pharmaceutically acceptable salts, or stereoisomers thereof.

17. The method of claim 1, wherein an antiviral therapy has been administered prior to the initial dose of the antibody.

18-19. (canceled)

20. An article of manufacture, comprising a container, a composition within the container comprising a PCSK9 antagonist antibody, and a package insert containing instructions to administer a therapeutically effective amount of the PCSK9 antagonist antibody for reducing a level of LDL-cholesterol in blood of a subject infected with HCV or at high risk of contracting HCV.
